(19) United States
(12) Patent Application Publication
(10) Pub. No.: US 2006/0148072 A1
(43) Pub. Date: Jul. 6, 2006

(54) Bioreactor system

(75) Inventors: Chun-Jen Liao, Taipei (TW); Yung-Chin Wu, Taipei County (TW); Ko-Chun Hsu, Kaohsiung City (TW); Hua-Chun Hsu, Miaoli County (TW); Chen-Chi Tsai, Taipei County (TW); Da-Pan Chen, Hsinchu City (TW)

Correspondence Address: BIRCH STEWART KOLASCH & BIRCH, PO BOX 747, FALLS CHURCH, VA, 22040-0747, US

(73) Assignee: INDUSTRIAL TECHNOLOGY RESEARCH INSTITUTE

(21) Appl. No.: 11/202,020

(22) Filed: Aug. 12, 2005

(30) Foreign Application Priority Data
Dec. 31, 2004 (TW) 93141731
Jun. 2, 2005 (TW) 94118164

(51) Int. Cl. C12M 1/00

(52) U.S. Cl. 435/290.1

(57) ABSTRACT

A bioreactor system. A supply container receives a cultivation solution. A bioreactor container is connected to the supply container and includes a porous carrier. The porous carrier carries a plurality of cells. The cultivation solution circulates between the supply container and the bioreactor container, providing needed nutrients to the cells.

### BACKGROUND

The invention relates to a bioreactor system, and in particular to a bioreactor system enabling continuous and closed operation of inoculation, cultivation, and harvest of anchorage-dependent cells.

Generally, tissue engineering reconstruction, stem cell clinical application, protein pharmaceutical production, and cell treatment require a considerable number of animal cells. Cultivation and production of sufficient cells thus play an important role in the aforementioned fields.

Accordingly, cultivation and production of cells, such as anchorage-dependent cells, can be accomplished both statically and dynamically. In static cultivation, the anchorage-dependent cells are directly inoculated into a porous carrier. The porous carrier is then placed in a Petri dish and a cultivation solution is filled therein. The anchorage-dependent cells can thus proliferate in the porous carrier. Nevertheless, because of the limited volume of a Petri dish, numerous Petri dishes are required for proliferation of the anchorage-dependent cells. Moreover, as inoculation of the anchorage-dependent cells, replacement of the cultivation solution, and harvest of the anchorage-dependent cells require manual operations, a number of operators and much cultivation space are required. Further, the anchorage-dependent cells are easily contaminated by the aforementioned manual operation and cultivation.

To avoid the aforementioned problems in static cultivation, the anchorage-dependent cells can be instead cultivated dynamically. Namely, high-density cultivation of the anchorage-dependent cells can be performed in a bioreactor capable of providing sufficient metabolism and exchange of nutrients within a cultivation solution.

Referring to FIG. 1, a conventional bioreactor 1 comprises a reaction tank 11, a cell carrier 12, a stir blade 13, a rotating shaft 14, a motor 15, an input pipe 16, and an output pipe 17. The cell carrier 12 is disposed in the reaction tank 11. The motor 15 is disposed on the reaction tank 11. The stir blade 13 is connected to the motor 15 by the rotating shaft 14 penetrating the cell carrier 12. The input pipe 16 and output pipe 17 are respectively connected to two opposing sides of the reaction tank 11.

Cultivation solution A and anchorage-dependent cells enter the reaction tank 11 via the input pipe 16. The cultivation solution A must completely cover the cell carrier 12. The stir blade 13 is driven by the motor 15 to stir the cultivation solution A in the reaction tank 11, attaching or inoculating the anchorage-dependent cells onto the cell carrier 12 and fully mixing the nutrients in the cultivation solution A. The anchorage-dependent cells can then proliferate in the cell carrier 12. After the anchorage-dependent cells are cultivated for a span of time, the cultivation solution A in the reaction tank 11 is discharged via the output pipe 17. At this point, the cell carrier 12 can be removed from the reaction tank 11 and enzyme, such as trypsin, can be applied to separate the anchorage-dependent cells from the cell carrier 12. Specifically, the cell carrier 12 is dissolved by the enzyme, such that the anchorage-dependent cells can be separated therefrom. The mixed solution including the anchorage-dependent cells and enzyme is then placed on a centrifuge, whereby the anchorage-dependent cells are separated from the enzyme. Accordingly, the proliferated anchorage-dependent cells can be obtained.

The bioreactor 1, however, has many drawbacks in cultivation of the anchorage-dependent cells (or cells). As the anchorage-dependent cells are inoculated in the cell carrier 12 by stirring, the inoculation rate thereof is reduced following increased volume of the reaction tank 11. To promote the inoculation rate of the anchorage-dependent cells, the proportion of the reaction tank 11 to the cell carrier 12 must be reduced. However, the reduced proportion of the reaction tank 11 to the cell carrier 12 indicates that the cultivation solution A is reduced, supplying few nutrients to the anchorage-dependent cells. Thus, the cultivation solution A must be frequently replaced. Moreover, to increase the amount of oxygen dissolved in the cultivation solution A, for the anchorage-dependent cells, the stirring speed of the stir blade 13 must be increased to enhance the exchange rate of air. The increased stirring speed of the stir blade 13, however, generates high shear force, causing the anchorage-dependent cells to separate from the cell carrier 12 and further death thereof. The proliferation of the anchorage-dependent cells is thus adversely affected. Furthermore, when the proliferated anchorage-dependent cells are harvested, the reaction tank 11 must be opened to remove the cell carrier 12, thereby contaminating the anchorage-dependent cells therein.

Hence, there is a need for a bioreactor system enabling continuous and closed operation of inoculation, cultivation, and harvest of cells, such as anchorage-dependent cells. Labor cost, and contamination of the cells (anchorage-dependent cells) can thus be reduced.

### SUMMARY

Accordingly, an exemplary embodiment of the invention provides a bioreactor system comprising a supply container and a bioreactor container. The supply container receives a cultivation solution. The bioreactor container is connected to the supply container and comprises a porous carrier. The porous carrier carries a plurality of cells. The cultivation solution circulates between the supply container and the bioreactor container, providing required nutrients to the cells.

The bioreactor system further comprises a motor disposed on the supply container.

The bioreactor system further comprises a stir blade disposed in the supply container and connected to the motor to agitate the cultivation solution.

The cells comprise anchorage-dependent cells.

The bioreactor system further comprises a first transportation pipe and a second transportation pipe respectively connected between the supply container and the bioreactor container. The cultivation solution flows from the supply container into the porous carrier of the bioreactor container via the first transportation pipe. The cultivation solution flows from the bioreactor container into the supply container via the second transportation pipe.

The bioreactor system further comprises a dissolution container connected to the first transportation pipe and receiving a dissolution. The dissolution flows into the porous carrier of the bioreactor container via the first transportation pipe, dissolving the porous carrier.

The bioreactor system further comprises a first peristaltic pump connected to the first transportation pipe. The cultivation solution flows into the porous carrier of the bioreactor container by operation of the first peristaltic pump.

The bioreactor system further comprises a second peristaltic pump connected to the second transportation pipe. The cultivation solution flows into the supply container by operation of the second peristaltic pump.

The bioreactor system further comprises a third transportation pipe connected between the first and second transportation pipes. The dissolution container is connected to the first transportation pipe between the bioreactor container and the third transportation pipe. The first peristaltic pump is connected to the first transportation pipe between the third transportation pipe and the supply container. The second peristaltic pump is connected to the second transportation pipe between the bioreactor container and the third transportation pipe.

The bioreactor system further comprises a first control valve, a second control valve, a third control valve, and a fourth control valve. The first control valve is connected to the first transportation pipe between the third transportation pipe and the first peristaltic pump. The second control valve is connected to the second transportation pipe between the third transportation pipe and the supply container. The third control valve is connected to the third transportation pipe. The fourth control valve is connected between the dissolution container and the first transportation pipe.

The bioreactor container further comprises a container body, an input pipe, and an output pipe. The porous carrier, input pipe, and output pipe are disposed in the container body. The porous carrier surrounds the input and output pipes. The input pipe is connected to the first transportation pipe and comprises a plurality of orifices on the pipe wall thereof. The output pipe is connected to the second transportation pipe. The cultivation solution flows into the porous carrier via the orifices of the input pipe. The cultivation solution flows out of the bioreactor container via the output pipe.

The output pipe is disposed in and extends outside the input pipe.

The orifices have different sizes and are uniformly formed on the pipe wall of the input pipe from smallest to largest.

The orifices have the same size and are formed on the pipe wall of the input pipe from sparse to dense.

The bioreactor container further comprises a container body, a first input pipe, a second input pipe, a third input pipe, and an output pipe. The porous carrier, first input pipe, second input pipe, third input pipe, and output pipe are disposed in the container body. The porous carrier surrounds the first input pipe, second input pipe, third input pipe, and output pipe. The caliber of the second input pipe is less than that of the first input pipe. The caliber of the third input pipe is less than that of the second input pipe. The first input pipe is connected to the first transportation pipe. The second input pipe is coaxially connected to the first input pipe. The third input pipe is coaxially connected to the second input pipe. The output pipe is connected to the second transportation pipe. The cultivation solution flows into the porous carrier via the first, second, and third input pipes. The cultivation solution flows out of the bioreactor container via the output pipe.

The output pipe is coaxially disposed in the first, second, and third input pipes and extends beyond the first and third input pipes.

The bioreactor system further comprises a sensor disposed in the supply container to detect the condition of the cultivation solution and concentration of oxygen dissolved therein.

The supply container further comprises an opening through which the cultivation solution and air flow into and out of the supply container.

The container body comprises a centrifugal tube.

The dissolution container comprises a syringe.

The porous carrier comprises alginate, N,O-carboxymethyl chitosan, or carboxymethyl cellulose.

The dissolution comprises EDTA (ethylenediminetetra acetic acid), sodium citriate, or EGTA (ethyleneglycol-bis (2-aminoethylether)-N′,N′,N′,N′-tetraactic acid).

### DESCRIPTION OF THE DRAWINGS

The invention can be more fully understood by reading the subsequent detailed description and examples with references made to the accompanying drawings, wherein:

FIG. 1 is a schematic plane view of a conventional bioreactor;

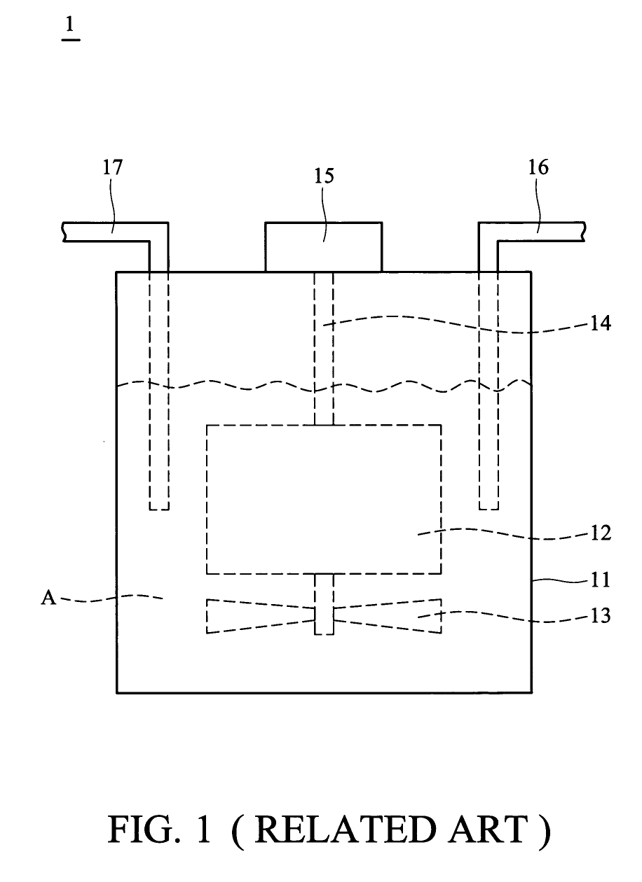

FIG. 2A is a schematic plane view of the bioreactor system of a first embodiment of the invention;

FIG. 2B is a schematic plane view of the bioreactor system of a second embodiment of the invention;

FIG. 3 is a schematic plane view of a bioreactor container applied to the bioreactor system of an embodiment of the invention;

FIG. 4 is a schematic plane view of another bioreactor container applied to the bioreactor system of an embodiment of the invention; and

FIG. 5 is a schematic plane view of yet another bioreactor container applied to the bioreactor system of an embodiment of the invention.

### DETAILED DESCRIPTION

### First Embodiment

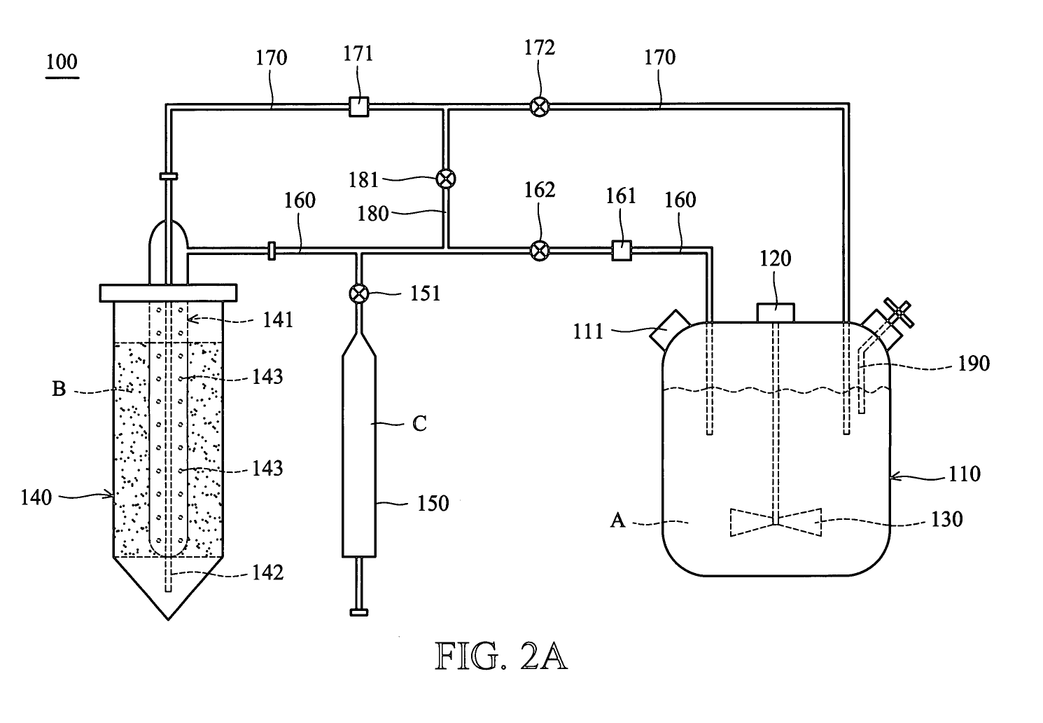

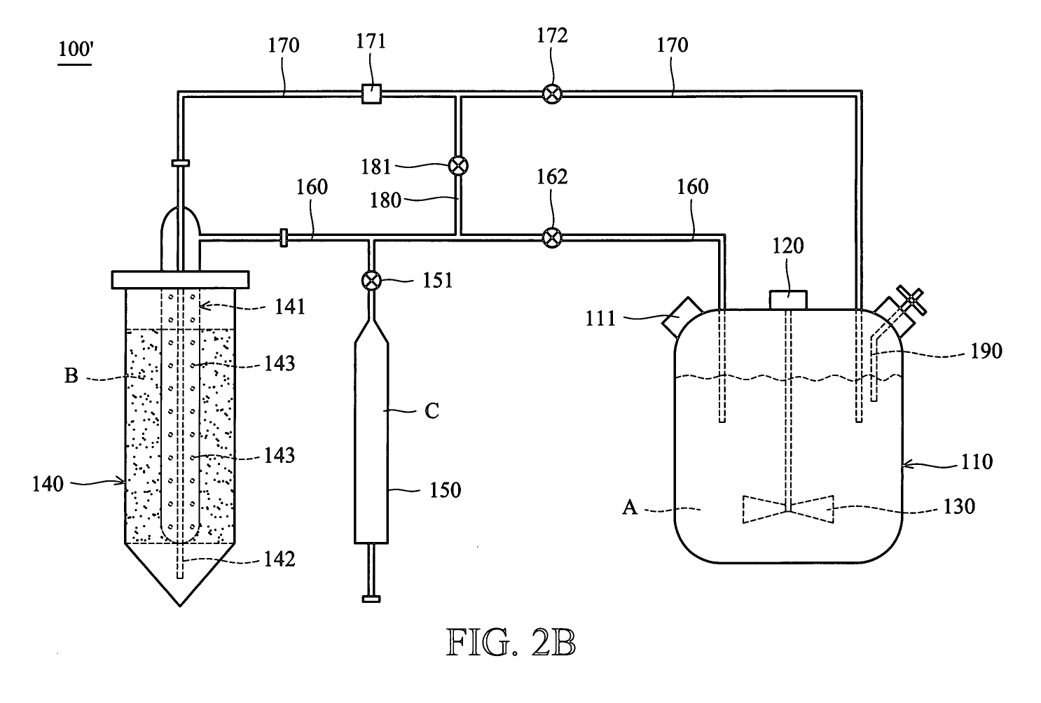

Referring to FIG. 2A, the bioreactor system 100 comprises a supply container 110, a motor 120, a stir blade 130, a bioreactor container 140, a dissolution container 150, a first transportation pipe 160, a second transportation pipe 170, a third transportation pipe 180, a first peristaltic pump 161, a second peristaltic pump 171, a first control valve 162, a second control valve 172, a third control valve 181, a fourth control valve 151, and a sensor 190.

As shown in FIG. 2A, the supply container 110 comprises an opening 111 through which a cultivation solution A and air are input to and output from the supply container 110. The motor 120 is disposed on the supply container 110. The stir blade 130 is disposed in the supply container 110 and is connected to the motor 120. The sensor 190 is disposed in the supply container 110, detecting the condition of the cultivation solution A and concentration of oxygen dissolved therein.

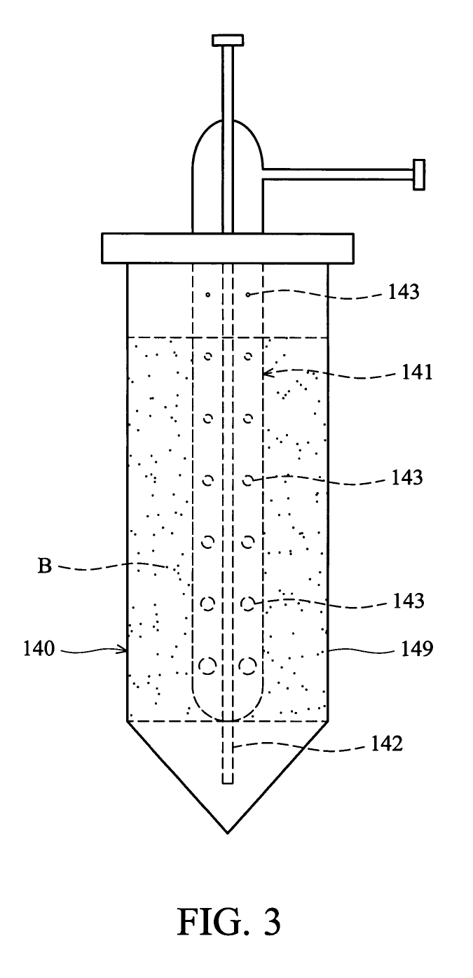

The bioreactor container 140 is connected to the supply container 110. Specifically, the bioreactor container 140 is connected to the supply container 110 by the first transportation pipe 160 and second transportation pipe 170. As shown in FIG. 2A and FIG. 3, the bioreactor container 140 comprises a porous carrier B, a container body 149, an input pipe 141, and an output pipe 142. The porous carrier B, input pipe 141, and output pipe 142 are disposed in the container body 149. The porous carrier B surrounds the input pipe 141 and output pipe 142. The input pipe 141 is connected to the first transportation pipe 160 and the output pipe 142 is connected to the second transportation pipe 170. Specifically, the output pipe 142 is disposed in and extends beyond the input pipe 141. Additionally, as shown in FIG. 3, the input pipe 141 comprises a plurality of orifices 143 on the pipe wall thereof. These orifices 143 have different sizes and are uniformly formed on the pipe wall of the input pipe 141 in order of increasing size. Namely, the orifices 143 on the upper part of the pipe wall of the input pipe 141 are smaller than those on the lower part thereof. Accordingly, the cultivation solution A uniformly flows out of the input pipe 141.

As shown in FIG. 2A, the third transportation pipe 180 is connected between the first transportation pipe 160 and the second transportation pipe 170. The dissolution container 150 is connected to the first transportation pipe 160 between the bioreactor container 140 and the third transportation pipe 180. The dissolution container 150 receives a dissolution C. The first peristaltic pump 161 is connected to the first transportation pipe 160 between the third transportation pipe 180 and the supply container 110. The second peristaltic pump 171 is connected to the second transportation pipe 170 between the bioreactor container 140 and the third transportation pipe 180. The first control valve 162 is connected to the first transportation pipe 160 between the third transportation pipe 180 and the first peristaltic pump 161. The second control valve 172 is connected to the second transportation pipe 170 between the third transportation pipe 180 and the supply container 110. The third control valve 181 is connected to the third transportation pipe 180. The fourth control valve 151 is connected between the dissolution container 150 and the first transportation pipe 160.

The container body 149 of the bioreactor container 140 may be a centrifugal tube, and the dissolution container 150 may be a syringe. The porous carrier B disposed in the bioreactor container 140 may comprise alginate, N,O-carboxymethyl chitosan, or carboxymethyl cellulose. The dissolution C may comprise EDTA (ethylenediminetetra acetic acid), sodium citriate, or EGTA (ethyleneglycol-bis (2-aminoethylether)-N′,N′,N′,N′-tetraactic acid).

The following description is directed to cultivation of anchorage-dependent cells using the bioreactor system 100.

As shown in FIG. 2A, after a cultivation solution containing a plurality of anchorage-dependent cells is input to the bioreactor container 140, the third control valve 181 is opened and the first control valve 162, second control valve 172, and fourth control valve 151 are closed. The second peristaltic pump 171 is then activated. At this point, the cultivation solution containing the anchorage-dependent cells circulates in the bioreactor container 140. Specifically, the cultivation solution containing the anchorage-dependent cells flows into the input pipe 141 of the bioreactor container 140 via the first transportation pipe 160. Then, the cultivation solution containing the anchorage-dependent cells uniformly perfuses the porous carrier B via the orifices 143 on the input pipe 141. Accordingly, the anchorage-dependent cells can be inoculated in caverns (not shown) of the porous carrier B. The cultivation solution flowing to the bottom of the bioreactor container 140 flows out via the output pipe 142 and second transportation pipe 170 and continues to circulate in the bioreactor container 140.

In another aspect, the motor 120 activates the stir blade 130. The cultivation solution A input to the supply container 110 via the opening 111 is agitated by the stir blade 130, aerating the cultivation solution A, increasing the concentration of oxygen dissolved therein.

After the anchorage-dependent cells are inoculated in the porous carrier B, the third control valve 181 and fourth control valve 151 remain closed and the first control valve 162 and second control valve 172 remain open. The first peristaltic pump 161 and second peristaltic pump 171 are simultaneously activated. At this point, the cultivation solution A circulates between the supply container 110 and the bioreactor container 140, continuously cultivating the anchorage-dependent cells in the porous carrier B of the bioreactor container 140. Specifically, the cultivation solution A in the supply container 110 flows into the input pipe 141 via the first transportation pipe 160. Then, the cultivation solution A uniformly flows into the porous carrier B via the orifices 143 on the input pipe 141, proliferating the anchorage-dependent cells. Moreover, the cultivation solution A containing metabolite of the anchorage-dependent cells flows to the bottom of the bioreactor container 140 and further into the supply container 110 via the output pipe 142 and second transportation pipe 170 to mix with the cultivation solution A therein. Accordingly, the anchorage-dependent cells in the porous carrier B are continuously supplied with the cultivation solution A containing needed nutrients and oxygen.

In another aspect, when nutrients in the cultivation solution A are insufficient, the cultivation solution A can be drawn out via the opening 111 of the supply container 110. Fresh cultivation solution A can then be input to the supply container 110 via the opening 111 thereof. Similarly, the fresh cultivation solution A circulates between the supply container 110 and the bioreactor container 140 to perfuse the porous carrier B and cultivate the anchorage-dependent cells therein.

After proliferation of the anchorage-dependent cells is complete, the first control valve 162, first peristaltic pump 161, third control valve 181, and fourth control valve 151 remain closed and the second control valve 172 and second peristaltic pump 171 remain open. At this point, the cultivation solution A in the bioreactor container 140 is drawn to the supply container 110. Then, the second control valve 172, second peristaltic pump 171, first control valve 162, third control valve 181, and first peristaltic pump 161 remain closed and the fourth control valve 151 remains open. The dissolution C in the dissolution container 150 is completely input to the bioreactor container 140 via the first transportation pipe 160 and input pipe 141. Then, the fourth control valve 151, first control valve 162, second control valve 172, and first peristaltic pump 161 remain closed and the second peristaltic pump 171 and third control valve 181 remain open. At this point, the dissolution C circulates in the bioreactor container 140 until the porous carrier B disposed therein is completely dissolved. The second peristaltic pump 171 and third control valve 181 are then closed and the bioreactor container 140 is separated from the first transportation pipe 160 and second transportation pipe 170. The bioreactor container 140 can be placed on a centrifuge and the proliferated anchorage-dependent cells are separated from the dissolution C thereby.

### Second Embodiment

Elements corresponding to those in the first embodiment share the same reference numerals.

Referring to FIG. 2B, this embodiment differs from the first embodiments in that the bioreactor system 100′ of this embodiment does not require any peristaltic pump (or the first peristaltic pump 161) connected to the first transportation pipe 160.

Structure, disposition, and function of other elements of the bioreactor system 100′ are the same as those of the bioreactor system 100, and explanation thereof is omitted for simplicity.

The following description is directed to cultivation of anchorage-dependent cells using the bioreactor system 100′.

As shown in FIG. 2B, after a cultivation solution containing a plurality of anchorage-dependent cells is input to the bioreactor container 140, the third control valve 181 is opened and the first control valve 162, second control valve 172, and fourth control valve 151 are closed. The second peristaltic pump 171 is then activated. At this point, the cultivation solution containing the anchorage-dependent cells circulates in the bioreactor container 140. Specifically, the cultivation solution containing the anchorage-dependent cells flows into the input pipe 141 of the bioreactor container 140 via the first transportation pipe 160. Then, the cultivation solution containing the anchorage-dependent cells uniformly perfuses the porous carrier B via the orifices 143 on the input pipe 141. Accordingly, the anchorage-dependent cells can be inoculated in caverns (not shown) of the porous carrier B. The cultivation solution flowing to the bottom of the bioreactor container 140 flows out via the output pipe 142 and second transportation pipe 170 and continues to circulate in the bioreactor container 140.

Similarly, the motor 120 activates the stir blade 130. The cultivation solution A input to the supply container 110 via the opening 111 is agitated by the stir blade 130, aerating the cultivation solution A, increasing the concentration of oxygen dissolved therein.

After the anchorage-dependent cells are inoculated in the porous carrier B, the third control valve 181 and fourth control valve 151 remain closed and the first control valve 162 and second control valve 172 remain open. The second peristaltic pump 171 is activated. At this point, the cultivation solution A circulates between the supply container 110 and the bioreactor container 140, continuously cultivating the anchorage-dependent cells in the porous carrier B of the bioreactor container 140. Specifically, the cultivation solution A in the supply container 110 flows into the input pipe 141 via the first transportation pipe 160. Then, the cultivation solution A uniformly flows into the porous carrier B via the orifices 143 on the input pipe 141, proliferating the anchorage-dependent cells. Moreover, the cultivation solution A containing metabolite of the anchorage-dependent cells flows to the bottom of the bioreactor container 140 and further into the supply container 110 via the output pipe 142 and second transportation pipe 170 to mix with the cultivation solution A therein. Accordingly, the anchorage-dependent cells in the porous carrier B are continuously supplied with the cultivation solution A containing needed nutrients and oxygen. Specifically, the bioreactor container 140 is connected outside by only the second transportation pipe 170 and first transportation pipe 160 and is completely sealed. Accordingly, as the bioreactor container 140 is completely sealed and has a fixed volume, the amount of the cultivation solution A flowing into the bioreactor container 140 via the second transportation pipe 170 is the same as that flowing out of the bioreactor container 140 via the first transportation pipe 160. When the third control valve 181 and fourth control valve 151 remain closed and the first control valve 162 and second control valve 172 remain open, the second transportation pipe 170, first transportation pipe 160, and bioreactor container 140 can be regarded as a closed system. Namely, as the inside volume of the bioreactor container 140 is fixed, the amount of cultivation solution A transported from the supply container 110 to the bioreactor container 140 by the second peristaltic pump 171 equals that expelled from the bioreactor container 140 to the supply container 110. Accordingly, the volume or height of the cultivation solution A in the bioreactor container 140 is thus fixed. Namely, the amount of the cultivation solution A in the bioreactor container 140 is not changed with time.

Similarly, when nutrients in the cultivation solution A are insufficient, the cultivation solution A can be drawn out via the opening 111 of the supply container 110. Fresh cultivation solution A can then be input to the supply container 110 via the opening 111 thereof. Similarly, the fresh cultivation solution A circulates between the supply container 110 and the bioreactor container 140 to perfuse the porous carrier B and cultivate the anchorage-dependent cells therein.

After proliferation of the anchorage-dependent cells is complete, the first control valve 162, third control valve 181, and fourth control valve 151 remain closed and the second control valve 172 and second peristaltic pump 171 remain open. At this point, the cultivation solution A in the bioreactor container 140 is drawn to the supply container 110. Then, the second control valve 172, second peristaltic pump 171, first control valve 162, and third control valve 181 remain closed and the fourth control valve 151 remains open. The dissolution C in the dissolution container 150 is completely input to the bioreactor container 140 via the first transportation pipe 160 and input pipe 141. Then, the fourth control valve 151, first control valve 162, and second control valve 172 remain closed and the second peristaltic pump 171 and third control valve 181 remain open. At this point, the dissolution C circulates in the bioreactor container 140 until the porous carrier B disposed therein is completely dissolved. The second peristaltic pump 171 and third control valve 181 are then closed and the bioreactor container 140 is separated from the first transportation pipe 160 and second transportation pipe 170. The bioreactor container 140 can be placed on a centrifuge and the proliferated anchorage-dependent cells are separated from the dissolution C thereby.

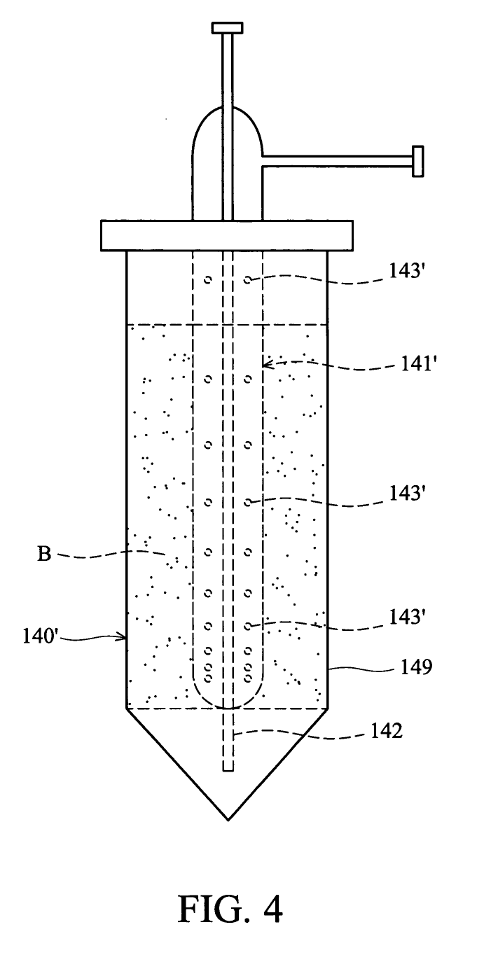

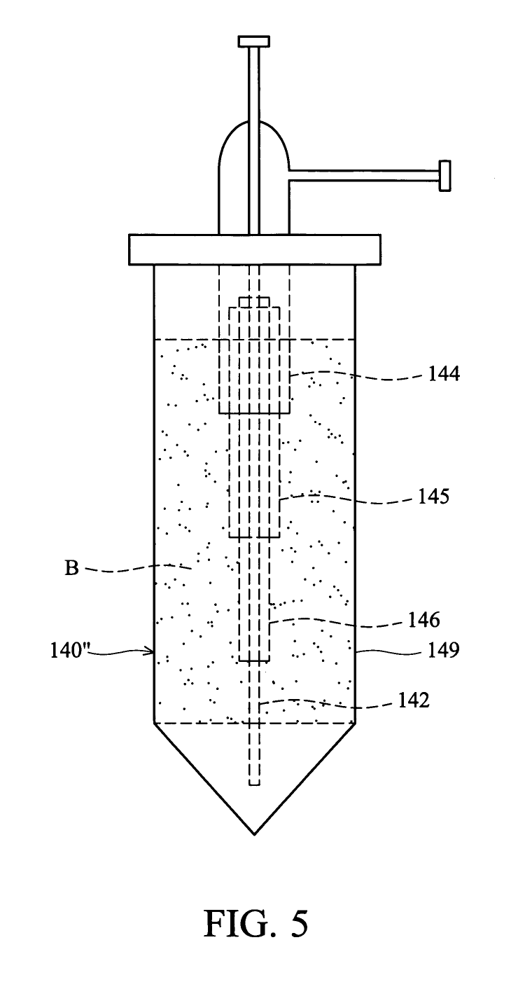

Moreover, in the aforementioned embodiments, the disclosed bioreactor container is not limited to the structure shown in FIG. 3. Namely, the bioreactor container may have the structure shown in FIG. 4 or FIG. 5 to enable proliferation of the anchorage-dependent cells.

As shown in FIG. 4, in a bioreactor container 140′, orifices 143′ on an input pipe 141′ have the same size and are formed on the pipe wall thereof from sparse to dense. Specifically, the vertical distance between the orifices 143′ on the upper part of the input pipe 141′ exceeds that on the lower part thereof. Elements corresponding to those in the bioreactor container 140 share the same reference numerals.

Accordingly, the input pipe 141′ and an output pipe 142 of the bioreactor container 140′ are respectively connected to the first transportation pipe 160 and second transportation pipe 170. The cultivation solution A uniformly flows into or perfuses the porous carrier B via the orifices 143′ on the input pipe 141′, proliferating the anchorage-dependent cells. Moreover, the cultivation solution A containing metabolite of the anchorage-dependent cells flows to the bottom of the bioreactor container 140′ and further flows into the supply container 110 via the output pipe 142 and second transportation pipe 170 to mix with the cultivation solution A therein.

As shown in FIG. 5, a bioreactor container 140″ comprises a container body 149, a first input pipe 144, a second input pipe 145, a third input pipe 146, and an output pipe 142. The first input pipe 144, second input pipe 145, third input pipe 146, and output pipe 142 are disposed in the container body 149. The porous carrier B surrounds the first input pipe 144, second input pipe 145, third input pipe 146, and output pipe 142. Specifically, the caliber of the second input pipe 145 is less than that of the first input pipe 144, and the caliber of the third input pipe 146 is less than that of the second input pipe 145. The second input pipe 145 is coaxially connected to the first input pipe 144. The third input pipe 146 is coaxially connected to the second input pipe 145. The output pipe 142 is coaxially disposed in the first input pipe 144, second input pipe 145, and third input pipe 146 and extends beyond the first input pipe 144 and third input pipe 146. Elements corresponding to those in the bioreactor container 140 share the same reference numerals.

Accordingly, the first input pipe 144 and output pipe 142 of the bioreactor container 140″ are respectively connected to the first transportation pipe 160 and second transportation pipe 170. The cultivation solution A uniformly flows into or perfuses the porous carrier B via the first input pipe 144, second input pipe 145, and third input pipe 146, proliferating the anchorage-dependent cells. Moreover, the cultivation solution A containing metabolite of the anchorage-dependent cells flows to the bottom of the bioreactor container 140″ and further into the supply container 110 via the output pipe 142 and second transportation pipe 170 to mix with the cultivation solution A therein.

In conclusion, the disclosed bioreactor system has the following advantages. As the anchorage-dependent cells are inoculated in the porous carrier B by continuous perfusion, the present bioreactor system provides enhanced inoculation rate of cells (anchorage-dependent cells) compared to conventional bioreactors. Additionally, as the present bioreactor system provides an independent supply container supplying the cultivation solution, the amount of cultivation solution required for proliferation of the cells (anchorage-dependent cells) is not limited by the capacity of the bioreactor container, thereby eliminating the need for frequent replacement of the cultivation solution. Moreover, the present bioreactor system continuously provides the cells (anchorage-dependent cells) with cultivation solution containing increased concentration of oxygen dissolved therein, thereby successfully proliferating the cells (anchorage-dependent cells). Furthermore, as inoculation, cultivation, and harvest of the cells (anchorage-dependent cells) are continuously performed in the closed bioreactor system, operation labor and costs and contamination of the cells (anchorage-dependent cells) are thus reduced.

While the invention has been described by way of example and in terms of preferred embodiment, it is to be understood that the invention is not limited thereto. To the contrary, it is intended to cover various modifications and similar arrangements (as would be apparent to those skilled in the art). Therefore, the scope of the appended claims should be accorded the broadest interpretation so as to encompass all such modifications and similar arrangements.

What is claimed is:

1. A bioreactor system, comprising:
a supply container receiving a cultivation solution; and
a bioreactor container connected to the supply container and comprising a porous carrier, wherein the porous carrier carries a plurality of cells, and the cultivation solution circulates between the supply container and the bioreactor container, providing needed nutrients to the cells.

2. The bioreactor system as claimed in claim 1, further comprising a motor disposed on the supply container.

3. The bioreactor system as claimed in claim 2, further comprising a stir blade disposed in the supply container and connected to the motor to agitate the cultivation solution.

4. The bioreactor system as claimed in claim 1, wherein the cells comprise anchorage-dependent cells.

5. The bioreactor system as claimed in claim 1, further comprising a first transportation pipe and a second transportation pipe respectively connected between the supply container and the bioreactor container, wherein the cultivation solution flows from the supply container into the porous carrier of the bioreactor container via the first transportation pipe, and the cultivation solution flows from the bioreactor container into the supply container via the second transportation pipe.

6. The bioreactor system as claimed in claim 5, further comprising a dissolution container connected to the first transportation pipe and receiving a dissolution, wherein the dissolution flows into the porous carrier of the bioreactor container via the first transportation pipe, dissolving the porous carrier.

7. The bioreactor system as claimed in claim 6, further comprising a first peristaltic pump connected to the first transportation pipe, wherein the cultivation solution flows into the porous carrier of the bioreactor container by operation of the first peristaltic pump.

8. The bioreactor system as claimed in claim 7, further comprising a second peristaltic pump connected to the second transportation pipe, wherein the cultivation solution flows into the supply container by operation of the second peristaltic pump.

9. The bioreactor system as claimed in claim 8, further comprising a third transportation pipe connected between the first and second transportation pipes, wherein the dissolution container is connected to the first transportation pipe between the bioreactor container and the third transportation pipe, the first peristaltic pump is connected to the first transportation pipe between the third transportation pipe and the supply container, and the second peristaltic pump is connected to the second transportation pipe between the bioreactor container and the third transportation pipe.

10. The bioreactor system as claimed in claim 9, further comprising a first control valve, a second control valve, a third control valve, and a fourth control valve, wherein the first control valve is connected to the first transportation pipe between the third transportation pipe and the first peristaltic pump, the second control valve is connected to the second transportation pipe between the third transportation pipe and the supply container, the third control valve is connected to the third transportation pipe, and the fourth control valve is connected between the dissolution container and the first transportation pipe.

11. The bioreactor system as claimed in claim 5, wherein the bioreactor container further comprises a container body, an input pipe, and an output pipe, the porous carrier, input pipe, and output pipe are disposed in the container body, the porous carrier surrounds the input and output pipes, the input pipe is connected to the first transportation pipe and comprises a plurality of orifices on the pipe wall thereof, the output pipe is connected to the second transportation pipe, the cultivation solution flows into the porous carrier via the orifices of the input pipe, and the cultivation solution flows out of the bioreactor container via the output pipe.

12. The bioreactor system as claimed in claim 11, wherein the output pipe is disposed in and extends beyond the input pipe.

13. The bioreactor system as claimed in claim 11, wherein the orifices have different sizes and are uniformly formed on the pipe wall of the input pipe in order of increasing size.

14. The bioreactor system as claimed in claim 11, wherein the orifices have the same size and are formed on the pipe wall of the input pipe from sparse to dense.

15. The bioreactor system as claimed in claim 5, wherein the bioreactor container further comprises a container body, a first input pipe, a second input pipe, a third input pipe, and an output pipe, the porous carrier, first input pipe, second input pipe, third input pipe, and output pipe are disposed in the container body, the porous carrier surrounds the first input pipe, second input pipe, third input pipe, and output pipe, the caliber of the second input pipe is less than that of the first input pipe, the caliber of the third input pipe is less than that of the second input pipe, the first input pipe is connected to the first transportation pipe, the second input pipe is coaxially connected to the first input pipe, the third input pipe is coaxially connected to the second input pipe, the output pipe is connected to the second transportation pipe, the cultivation solution flows into the porous carrier via the first, second, and third input pipes, and the cultivation solution flows out of the bioreactor container via the output pipe.

16. The bioreactor system as claimed in claim 15, wherein the output pipe is coaxially disposed in the first, second, and third input pipes and extends beyond the first and third input pipes.

17. The bioreactor system as claimed in claim 1, further comprising a sensor disposed in the supply container to detect the condition of the cultivation solution and concentration of oxygen dissolved therein.

18. The bioreactor system as claimed in claim 1, wherein the supply container further comprises an opening through which the cultivation solution and air flow into and out of the supply container.

19. The bioreactor system as claimed in claim 11, wherein the container body comprises a centrifugal tube.

20. The bioreactor system as claimed in claim 15, wherein the container body comprises a centrifugal tube.

21. The bioreactor system as claimed in claim 6, wherein the dissolution container comprises a syringe.

22. The bioreactor system as claimed in claim 1, wherein the porous carrier comprises alginate, N,O-carboxymethyl chitosan, or carboxymethyl cellulose.

23. The bioreactor system as claimed in claim 6, wherein the dissolution comprises EDTA (ethylenediminetetra acetic acid), sodium citriate, or EGTA (ethyleneglycol-bis (2-aminoethylether)-N′,N′,N′,N′-tetraactic acid).

24. A bioreactor container, comprising:
a container body;
an input pipe disposed in the container body and comprising a plurality of orifices on the pipe wall thereof;
an output pipe disposed in the container body; and
a porous carrier disposed in the container body and surrounding the input and output pipes.

25. The bioreactor container as claimed in claim 24, wherein the output pipe is disposed in and extends beyond the input pipe.

26. The bioreactor container as claimed in claim 24, wherein the orifices have different sizes and are uniformly formed on the pipe wall of the input pipe in order of increasing size.

27. The bioreactor container as claimed in claim 24, wherein the orifices have the same size and are formed on the pipe wall of the input pipe from sparse to dense.

28. The bioreactor container as claimed in claim 24, wherein the container body comprises a centrifugal tube.

29. A bioreactor container, comprising:
a container body;
a first input pipe disposed in the container body;
a second input pipe disposed in the container body and coaxially connected to the first input pipe;
a third input pipe disposed in the container body and coaxially connected to the second input pipe, wherein the caliber of the second input pipe is less than that of the first input pipe, and the caliber of the third input pipe is less than that of the second input pipe;
an output pipe disposed in the container body; and
a porous carrier disposed in the container body and surrounding the first input pipe, second input pipe, third input pipe, and output pipe.

30. The bioreactor container as claimed in claim 29, wherein the output pipe is coaxially disposed in the first, second, and third input pipes and extends beyond the first and third input pipes.

31. The bioreactor container as claimed in claim 29, wherein the container body comprises a centrifugal tube.

32. A bioreactor container, comprising:
a supply container receiving a cultivation solution;
a bioreactor container comprising a porous carrier carrying a plurality of cells;
a first transportation pipe connected between the supply container and the bioreactor container; and
a second transportation pipe connected between the supply container and the bioreactor container, wherein the cultivation solution flows from the supply container into the porous carrier of the bioreactor container via the first transportation pipe, the cultivation solution flows from the bioreactor container into the supply container via the second transportation pipe, and the cultivation solution circulates between the supply container and the bioreactor container, providing needed nutrients to the cells.

33. The bioreactor system as claimed in claim 32, further comprising a motor disposed on the supply container.

34. The bioreactor system as claimed in claim 33, further comprising a stir blade disposed in the supply container and connected to the motor to agitate the cultivation solution.

35. The bioreactor system as claimed in claim 32, wherein the cells comprise anchorage-dependent cells.

36. The bioreactor system as claimed in claim 32, further comprising a dissolution container connected to the first transportation pipe and receiving a dissolution, wherein the dissolution flows into the porous carrier of the bioreactor container via the first transportation pipe, dissolving the porous carrier.

37. The bioreactor system as claimed in claim 32, further comprising a second peristaltic pump connected to the second transportation pipe, wherein the cultivation solution flows into the supply container and porous carrier of the bioreactor container by operation of the second peristaltic pump.

38. The bioreactor system as claimed in claim 37, further comprising a third transportation pipe connected between the first and second transportation pipes, wherein the dissolution container is connected to the first transportation pipe between the bioreactor container and the third transportation pipe, and the second peristaltic pump is connected to the second transportation pipe between the bioreactor container and the third transportation pipe.

39. The bioreactor system as claimed in claim 38, further comprising a first control valve, a second control valve, a third control valve, and a fourth control valve, wherein the first control valve is connected to the first transportation pipe between the third transportation pipe and the supply container, the second control valve is connected to the second transportation pipe between the third transportation pipe and the supply container, the third control valve is connected to the third transportation pipe, and the fourth control valve is connected between the dissolution container and the first transportation pipe.

40. The bioreactor system as claimed in claim 32, wherein the bioreactor container further comprises a container body, an input pipe, and an output pipe, the porous carrier, input pipe, and output pipe are disposed in the container body, the porous carrier surrounds the input and output pipes, the input pipe is connected to the first transportation pipe and comprises a plurality of orifices on the pipe wall thereof, the output pipe is connected to the second transportation pipe, the cultivation solution flows into the porous carrier via the orifices of the input pipe, and the cultivation solution flows out of the bioreactor container via the output pipe.

41. The bioreactor system as claimed in claim 40, wherein the output pipe is disposed in and extends beyond the input pipe.

42. The bioreactor system as claimed in claim 40, wherein the orifices have different sizes and are uniformly formed on the pipe wall of the input pipe in order of increasing size.

43. The bioreactor system as claimed in claim 40, wherein the orifices have the same size and are formed on the pipe wall of the input pipe from sparse to dense.

44. The bioreactor system as claimed in claim 32, wherein the bioreactor container further comprises a container body, a first input pipe, a second input pipe, a third input pipe, and an output pipe, the porous carrier, first input pipe, second input pipe, third input pipe, and output pipe are disposed in the container body, the porous carrier surrounds the first input pipe, second input pipe, third input pipe, and output pipe, the caliber of the second input pipe is less than that of the first input pipe, the caliber of the third input pipe is less than that of the second input pipe, the first input pipe is connected to the first transportation pipe, the second input pipe is coaxially connected to the first input pipe, the third input pipe is coaxially connected to the second input pipe, the output pipe is connected to the second transportation pipe, the cultivation solution flows into the porous carrier via the first, second, and third input pipes, and the cultivation solution flows out of the bioreactor container via the output pipe.

45. The bioreactor system as claimed in claim 44, wherein the output pipe is coaxially disposed in the first, second, and third input pipes and extends beyond the first and third input pipes.

46. The bioreactor system as claimed in claim 32, further comprising a sensor disposed in the supply container to detect the condition of the cultivation solution and concentration of oxygen dissolved therein.

47. The bioreactor system as claimed in claim 32, wherein the supply container further comprises an opening through which the cultivation solution and air flow into and out of the supply container.

48. The bioreactor system as claimed in claim 40, wherein the container body comprises a centrifugal tube.

49. The bioreactor system as claimed in claim 44, wherein the container body comprises a centrifugal tube.

50. The bioreactor system as claimed in claim 36, wherein the dissolution container comprises a syringe.

51. The bioreactor system as claimed in claim 32, wherein the porous carrier comprises alginate, N,O-carboxymethyl chitosan, or carboxymethyl cellulose.

52. The bioreactor system as claimed in claim 36, wherein the dissolution comprises EDTA (ethylenediminetetra acetic acid), sodium citriate, or EGTA (ethyleneglycol-bis (2-aminoethylether)-N′,N′,N′,N′-tetraactic acid).
